(19) United States
(12) Patent Application Publication
(10) Pub. No.: US 2023/0175715 A1
(43) Pub. Date: Jun. 8, 2023

(54) INTERACTIVE NIGHTLIGHT WITH AIR CLEANING

(71) Applicant: Carrier Corporation, Palm Beach Gardens, FL (US)

(72) Inventors: Blake Smith, Concord, NC (US); Kenny Abell, Indianapolis, IN (US); Chrystal Jolliffe, London, OH (US); Alissa D. Doose, Denver, CO (US); Michael J. Birnkrant, Manlius, NY (US); Bridget Volz, Lizton, IN (US)

(21) Appl. No.: 18/074,768

(22) Filed: Dec. 5, 2022

Related U.S. Application Data
(60) Provisional application No. 63/286,279, filed on Dec. 6, 2021.

(51) Int. Cl. F24F 6/02; F21V 33/00; B01D 46/00

(52) U.S. Cl. CPC F24F 6/02; F21V 33/0088; B01D 46/0027; B01D 2279/40

(57) ABSTRACT

An environmental control device including: a base housing; a topper configured to be attachable and detachable from the base housing; one or more environmental systems; and one or more environmental sensors, wherein the topper is shaped as a figurine.

### CROSS-REFERENCE TO RELATED APPLICATIONS

This application claims the benefit of U.S. provisional patent application Ser. No. 63/286,279, filed Dec. 6, 2021, the entire contents of which are incorporated herein by reference.

### BACKGROUND

The embodiments herein generally relate to generally relates to room environmental control devices.

Conventional environmental control devices for children's rooms are large and typically have limited functionality. They are often not always aesthetically pleasing from a child's perspective and look out of place with children's décor.

### BRIEF DESCRIPTION

According to one embodiment, an environmental control device is provided. The environmental control device including: a base housing; a topper configured to be attachable and detachable from the base housing; one or more environmental systems; and one or more environmental sensors, wherein the topper is shaped as a figurine.

In addition to one or more of the features described above, or as an alternative, further embodiments of the transport refrigeration system may include that the base housing is translucent or transparent.

In addition to one or more of the features described above, or as an alternative, further embodiments of the transport refrigeration system may include that the top is translucent or transparent.

In addition to one or more of the features described above, or as an alternative, further embodiments of the transport refrigeration system may include that the one or more environmental systems include at a least one of: an air filtration system; a night light; a humidifier; a dehumidifier; a speaker; or a noise machine.

In addition to one or more of the features described above, or as an alternative, further embodiments of the transport refrigeration system may include that the one or more environmental systems include: an air filtration system; a night light; a humidifier; a dehumidifier; and a speaker or a noise machine.

In addition to one or more of the features described above, or as an alternative, further embodiments of the transport refrigeration system may include that the one or more environmental sensors include at a least one of: an air quality sensor; a light sensor; a humidity sensor; a microphone; a video camera; or a thermometer.

In addition to one or more of the features described above, or as an alternative, further embodiments of the transport refrigeration system may include that the one or more environmental sensors include: an air quality sensor; a light sensor; a humidity sensor; a microphone; a video camera; and a thermometer.

In addition to one or more of the features described above, or as an alternative, further embodiments of the transport refrigeration system may include that the one or more environmental systems include a humidifier, wherein the topper further includes a stationary component, and wherein the humidifier is configured to direct humidified air from the humidifier through the stationary component of the topper to make it appear that smoke is emanating from the stationary component of the topper.

In addition to one or more of the features described above, or as an alternative, further embodiments of the transport refrigeration system may include that an airflow is generated by the one or more environmental system, wherein the topper further includes a motive component, and wherein the airflow is directed to the motive component of the topper to move the motive component.

In addition to one or more of the features described above, or as an alternative, further embodiments of the transport refrigeration system may include that an airflow is generated by the one or more environmental system, wherein the topper further includes a motive component an orifice, and wherein the airflow is directed through the orifice at the motive component of the topper to move the motive component.

According to another embodiment, an environmental control system is provided. The environmental control system including: a computer application installed on a computing device or accessible via the computing device; an environmental control device in communication with the computing device, the environmental control device including: a base housing; a topper configured to be attachable and detachable from the base housing; one or more environmental systems, the one or more environmental systems being configured to receive commands from the computer application; and one or more environmental sensors, the one or more environmental sensors being configured to transmit data to the computer application, wherein the topper is shaped as a figurine.

In addition to one or more of the features described above, or as an alternative, further embodiments of the transport refrigeration system may include that the base housing is translucent or transparent.

In addition to one or more of the features described above, or as an alternative, further embodiments of the transport refrigeration system may include that the top is translucent or transparent.

In addition to one or more of the features described above, or as an alternative, further embodiments of the transport refrigeration system may include that the one or more environmental systems include at a least one of: an air filtration system; a night light; a humidifier; a dehumidifier; a speaker; or a noise machine.

In addition to one or more of the features described above, or as an alternative, further embodiments of the transport refrigeration system may include that the one or more environmental systems include: an air filtration system; a night light; a humidifier; a dehumidifier; and a speaker or a noise machine.

In addition to one or more of the features described above, or as an alternative, further embodiments of the transport refrigeration system may include that the one or more environmental sensors include at a least one of: an air quality sensor; a light sensor; a humidity sensor; a microphone; a video camera; or a thermometer.

In addition to one or more of the features described above, or as an alternative, further embodiments of the transport refrigeration system may include that the one or more environmental sensors include: an air quality sensor; a light sensor; a humidity sensor; a microphone; a video camera; and a thermometer.

In addition to one or more of the features described above, or as an alternative, further embodiments of the transport refrigeration system may include that the one or more environmental systems include a humidifier, wherein the topper further includes a stationary component, and wherein the humidifier is configured to direct humidified air from the humidifier through the stationary component of the topper to make it appear that smoke is emanating from the stationary component of the topper.

In addition to one or more of the features described above, or as an alternative, further embodiments of the transport refrigeration system may include that an airflow is generated by the one or more environmental system, wherein the topper further includes a motive component, and wherein the airflow is directed to the motive component of the topper to move the motive component.

In addition to one or more of the features described above, or as an alternative, further embodiments of the transport refrigeration system may include that an airflow is generated by the one or more environmental system, wherein the topper further includes a motive component an orifice, and wherein the airflow is directed through the orifice at the motive component of the topper to move the motive component.

Technical effects of embodiments of the present disclosure include incorporating an interchangeable figurine as a topper for an environment control device for a child's room.

The foregoing features and elements may be combined in various combinations without exclusivity, unless expressly indicated otherwise. These features and elements as well as the operation thereof will become more apparent in light of the following description and the accompanying drawings. It should be understood, however, that the following description and drawings are intended to be illustrative and explanatory in nature and non-limiting.

### BRIEF DESCRIPTION OF THE DRAWINGS

The following descriptions should not be considered limiting in any way. With reference to the accompanying drawings, like elements are numbered alike:

FIG. 1 is a schematic illustration of an exemplary environmental control system for a child's bedroom, according to an embodiment of the present disclosure;

FIG. 2 is a schematic illustration of an exemplary environmental control device, according to an embodiment of the present disclosure;

FIG. 3 is a schematic illustration of an exemplary topper for an environmental control device, according to an embodiment of the present disclosure;

FIG. 4 is a schematic illustration of an exemplary topper for an environmental control device, according to an embodiment of the present disclosure; and

FIG. 5 is a schematic illustration of an exemplary environmental control system for a child's bedroom, according to an embodiment of the present disclosure.

### DETAILED DESCRIPTION

A detailed description of one or more embodiments of the disclosed apparatus and method are presented herein by way of exemplification and not limitation with reference to the Figures.

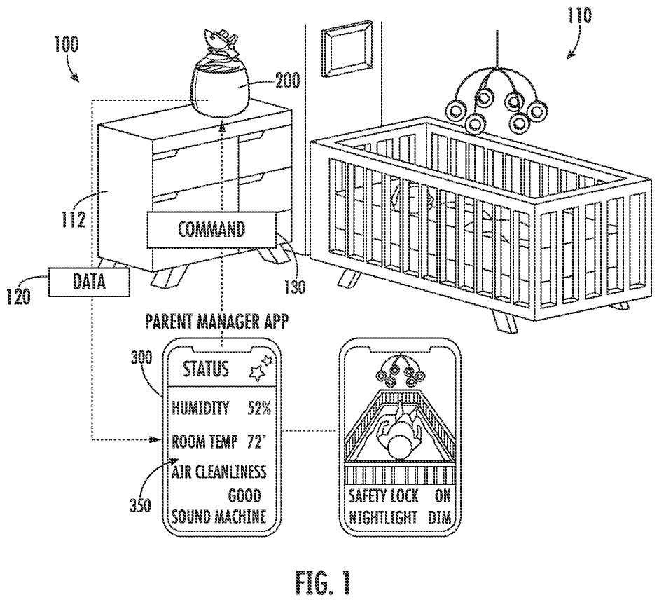

Referring now to FIG. 1, a schematic illustration of an environmental control system 100 for a child's bedroom 110 is illustrated, in accordance with an embodiment of the present disclosure. It is understood that while the environmental control system 100 is illustrated in relation to a child's bedroom 110, the environmental control system 100 described herein may be applicable to and utilized in any room within a home or building. The environmental control system 100 includes an environmental control device 200 and an associated computer application 350 that may be installed on a computing device 300 or accessible through the computing device 300 (e.g., software as a service)

The computing device 300 may be a desktop computer, a laptop computer, or a mobile computing device that is typically carried by a person, such as, for example a phone, a smart phone, a PDA, a smart watch, a tablet, a laptop, or any other mobile computing device known to one of skill in the art.

The environmental control device 200 may be equipped with one or more environmental systems to adjust an environment within the child's bedroom 110. The environmental systems may include an air filtration system, a night light, a humidifier, a dehumidifier, speaker, and/or a noise machine, as discussed further herein. The environmental control device 200 may be equipped with one or more environmental sensors to monitor an environment with the children's bedroom 110. The environmental sensors may include an air quality sensor, a microphone, a video camera, a humidity sensor, a thermometer, and a light sensor. The environmental sensors are configured to monitor environmental conditions and compile the environmental conditions monitored as data 120.

The environmental control device 200 is in communication with the computer application 350 and may be configured to transmit the data 120 to the computing device 300 either directly or through a network, such as, for example, the internet. The data 120 may be viewable through the application 350 and may display and/or provide audio of environmental conditions in the room, including, but not limited to, humidity, temperature, air cleanliness, sound, video, a safety lock status, and a night light status. The safety lock status is a safety feature that ensures a child cannot disturb the settings on the environmental control device 200 and/or remove any component associated with the environmental control device 200, such as, for example, the topper 210. The safety lock status as displayed on the application 350 may also reflect the status of whether the environmental control device 200 is “locked” or “unlocked”, such as, for example, to fill the humidifier with water.

A person may be able to monitor data 120 via the application 350 and transmit a command 130 through the application 350 from the computing device 300 to the environmental control device 200. The command 130 may adjust operation of an environmental system of the environmental control device 200.

As illustrated in FIG. 1, the environmental control device 200 is compact and configured to fit easily on a dresser 112 or nightstand within the child's bedroom 110. Advantageously, the environmental control device 200 is configured to replace multiple different separate devices, including, but not limited to a baby monitor camera, a sound machine, a night light, a humidifier, a dehumidifier, and an air filtration system, which typically take up a great deal of space on the dresser 112 and clutter up the room overall. Advantageously, since the environmental control device 200 replaces these multiple different devices, room is saved on the dresser 112 and the child's bedroom 110 is decluttered.

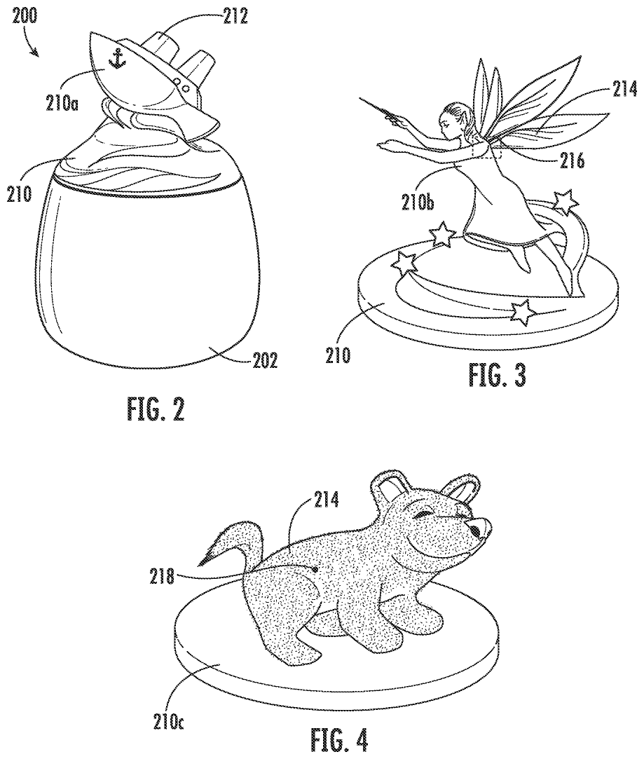

Referring now to FIGS. 2-4, with continued reference to FIG. 1, a schematic illustration of an environmental control device 200 is illustrated, in accordance with an embodiment of the present disclosure. As illustrated in FIG. 2, the environmental control device 200 includes a base housing 202 and a topper 210. The topper 210 is attachable and detachable from the base housing 202. The topper 210 is configured to fit on top of the base housing 202. The topper 210 may be removably secured to the base housing 202 through an interlocking device, such as, for example, interlocking threads or interlocking components that interlock when pushed together. In one example, the interlocking device may be interlocking threads (not shown) and the topper 210 may be screwed into the base housing 202 to temporarily secure the topper 210 to the base housing 202. In another example, the interlocking device may be interlocking components that interlock when pushed together and the topper 210 may be pushed onto the base housing 202 and the topper 210 may snap into the base housing 202 to temporarily secure the topper 210 to the base housing 202. Alternatively, the topper 210 may simply rest on top of the base housing 202. The topper 210 is configured to be interchangeable with other base housings 202 and the base housing 202 is configured to be interchangeable with other toppers 210. Advantageously, since the topper 210 is easily removably from the base housing 202, this allows different toppers 210 to be interchanged with the base housing 202 in order to provide different aesthetics and/or function to the overall environmental control device 200. In one example, the topper 210 may be configured to be easily removable to provide easy access to water reservoir or filter of a humidifier. The topper 210 may have a hidden attachment mechanism that does not disrupt the design. The topper 210 may have a child lock feature that is configured to lock the topper in place to avoid removable by a child, while being easily removable by an adult.

The topper 210 may be shaped as a figurine. The topper 210 may be a boat topper 210a that is shaped like a boat, a fairy topper 210b that is shaped like a fairy, or a puppy topper 210c that is shaped like a puppy. It is understood that the topper 210 is not limited to the boat topper 210a, the fairy topper 210b, and the puppy topper 210c, and the topper 210 may have any conceivable shape, color, and/or design.

As will be discussed further herein, one or more environmental systems within the base housing 202 may generate an airflow. For example, a humidifier, dehumidifier, and/or an air filtration system may generate an outlet airflow. The airflow generated by the one or more environmental systems may be directed through the topper 210 to produce an artistic effect or animate the topper 210. In an embodiment, the airflow generated by the one or more environmental systems may be directed through and out of a stationary component 212 of the topper 210 to make it appear that smoke is emanating from the stationary component 212 of the topper 210. For example, the stationary component 212 may be a smokestack of the boat topper 210a and humidified air may be directed through the smokestack and exit the smokestack to make it appear that the boat topper 210a is an actual boat. Similarly, the effect may be utilized if the topper 210 was a train with humidified air coming from the steam dome of the train or a dragon with humidified air coming from the mouth of the dragon

In an embodiment, the one or more environmental systems may be configured to generate an airflow. The airflow generated by the one or more environmental systems may be directed through a turbine 216 or similar device in the topper and out of the topper to actuate a motive component 214 of the topper 210 or to move the motive component 214. For example, the motive component 214 may be wings of the fairy of the fairy topper 210b, and the humified air or air from the filtration system may be directed through a turbine 216 that is operably connected to wings of the fairy and configured to move the wings of the fairy when rotated by the air. In another example, the humified air or air from the filtration system may be utilized if the topper 20 was an airplane to rotate propellers of the airplane, if the topper 20 was a helicopter to rotate rotors of the helicopter, or if the topper 20 was a windmill to rotate the blades of the windmill. The airflow generated by the one or more environmental systems may be directed to cause randomized motion. For example, the airflow would cause items to move about randomly as if in a snow globe or fishbowl.

In an embodiment, the airflow generated by the one or more environmental systems may be directed through and out of an orifice 218 in the topper 210 to actuate a motive component 214 of the topper 210 or to move the motive component 214. The orifice 218 may be configured to direct air directly at the motive component 214 to actuate the motive component 214 of the topper 210 or to move the motive component 214. For example, the motive component 214 may be hair on a puppy of the puppy topper 210c, and the humified air or air from the filtration system may be directed through the orifice 218 that is directed at the hair of the puppy to move the hair. In another example, the humified air or air from the filtration system may be utilized if the topper 20 was a parachute to raise or lower the parachute or if the topper 20 was a hot air balloon to raise or lower the hot air balloon.

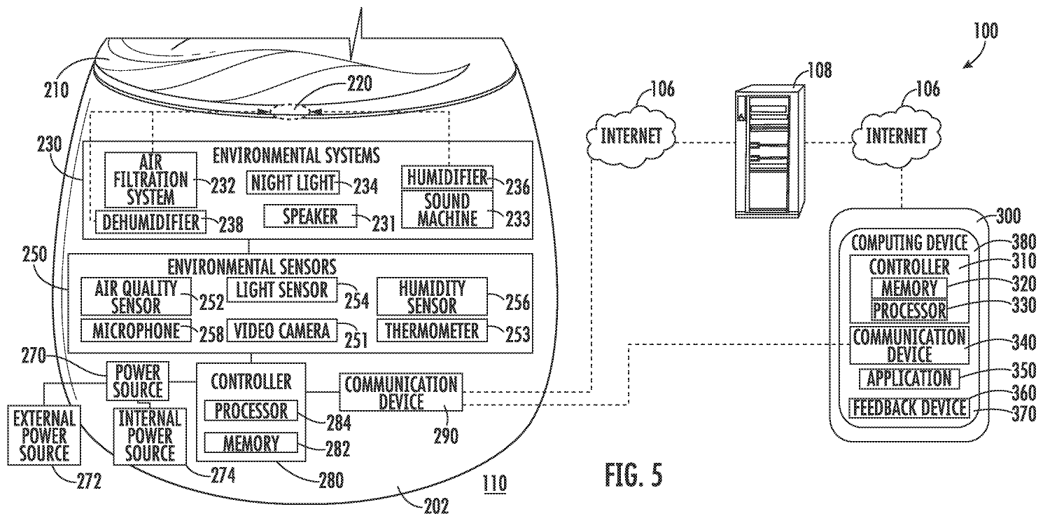

Referring now to FIG. 5, with continued reference to FIGS. 1-4, a schematic illustration of an environmental control system 100 is illustrated, in accordance with an embodiment of the present disclosure. It should be appreciated that, although particular systems are separately defined in the schematic block diagrams, each or any of the systems may be otherwise combined or separated via hardware and/or software.

As aforementioned, the topper 210 is removable from the base housing 202 and interchangeable with other toppers 210, thus a majority of the components of the environmental control device 200 may be located within the base housing 202. However, the embodiments disclosed herein are still applicable to environmental control devices 200 with environmental system 230 and environmental sensors 250 located in the topper 210. The base housing 202 may contain the environmental systems 230, the environmental sensors 250, a controller 280, and a communication device 290. In embodiment, the base housing 202 may contain all of the environmental systems 230, all of the environmental sensors 250, the controller 280, and the communication device 290.

The environmental systems 230 may include an air filtration system 232, a night light 234, a humidifier 236, a dehumidifier 238, a speaker 231, and/or a noise machine 233. The air filtration system 232 is configured to intake air from the child's bedroom 110, filter the air, and then return the air back to the room through an outlet 220. The outlet 220 may flow through the topper 210 as aforementioned. The humidifier 236 may include a water tank (not shown) and is configured to intake air from the bedroom 110, add water vapor to the air by evaporating water in the water tank, and then return the air with the added vapor as humidified air back to the room through the outlet 220. The dehumidifier 238 may include a water reservoir (not shown) and is configured to intake air from the bedroom 110, extract (e.g., condense) water from the air, store the extracted water in the reservoir, and then return the air without the water as dehumidified air back to the room through the outlet 220. The outlet 220 is configured to be direct the air into the topper 210.

The night light 234 may be configured to generate a light to be emitted from the environmental control device 200. The environmental control device 200 may be configured to emit the light from the night through the topper 210 and/or the base housing 202. A portion or all of the topper 210 may be transparent to allow light to transfer through. A portion or all of the topper 210 may be translucent to allow light to transfer through. A portion or all of the base housing 202 may be transparent to allow light to transfer through. A portion or all of base housing 202 may be translucent to allow light to transfer through. The night light 234 may be able to vary an intensity or a color of the light generated. For example, the night light 234 may brighten slowly to wake up a child in the morning. In another example, the night light 234 may be softer at night to not keep the baby awake.

The speaker 231 may be configured to generate a sound to be emitted from the environmental control device 200. The sound may be white noise, music, or audio transmitted from a user of the computer application 350. The sound machine 233 may be configured to generate a sound to be emitted from the environmental control device 200. The sound machine 333 may be configured to generate the sound through a mechanical means, such as, for example, a fan. The sound machine 233 may be necessary with the speaker 231.

The environmental sensors 250 may include an air quality sensor 252, a light sensor 254, a humidity sensor 256, a microphone 258, a video camera 251, and a thermometer 253.

The air quality sensor 252 may be configured to detect a quality of the air within the bedroom 110. The air quality sensor 252 may be configured to detect levels of various particulates and pollutants in the air within the bedroom 110. The air quality sensor 252 may be configured to detect temperature, humidity, PM2.5 (e.g., particulate count), TVOCs (Total Volatile Organic Compounds), CO, and/or CO2.

The light sensor 254 may be configured to detect a level of light within the bedroom 110. The humidity sensor 256 may be configured to detect a humidity level within the bedroom 110. The microphone 258 may be configured to detect sound within the bedroom 110. The video camera 251 may be configured to capture video within the bedroom 110. The thermometer 253 may be configured to detect a temperature within the bedroom 110.

The power source 270 may include an internal power source 274 and may be electrically connected to an external power source 272. The internal power source 274 may be a battery, capacity, or any other energy storage device known to one of skill of the art. The external power source 272 may be a standard wall outlet. The internal power source 274 may be charged by the external power source 272. The environmental control device 200 may be powered by electricity from the external power source 272 and/or the internal power source 274.

The environmental control device 200 also includes a controller 280 configured to control operations of the environmental control device 200. The controller 280 may be an electronic controller including a processor 284 and an associated memory 282 comprising computer-executable instructions (i.e., computer program product) that, when executed by the processor 284, cause the processor 284 to perform various operations. The processor 284 may be, but is not limited to, a single-processor or multi-processor system of any of a wide array of possible architectures, including field programmable gate array (FPGA), central processing unit (CPU), application specific integrated circuits (ASIC), digital signal processor (DSP) or graphics processing unit (GPU) hardware arranged homogenously or heterogeneously. The memory 282 may be but is not limited to a random access memory (RAM), read only memory (ROM), or other electronic, optical, magnetic or any other computer readable medium.

The environmental control device 200 also includes a communication device 290. The communication device 290 may be wireless communication including but not limited to Wi-Fi, Bluetooth, Zigbee, Sub-GHz RF Channel, cellular, or any other wireless signal known to one of skill in the art. The communication device 290 may be configured to communicate directly with the computing device 300. Alternatively, or additionally, the communication device 290 may be configured to communicate with the computing device 300 through the internet 106 and/or a remote server 108.

The computing device 300 includes a controller 310 configured to control operations of the computing device 300. The controller 310 may be an electronic controller including a processor 330 and an associated memory 320 comprising computer-executable instructions (i.e., computer program product) that, when executed by the processor 330, cause the processor 330 to perform various operations. The processor 330 may be, but is not limited to, a single-processor or multi-processor system of any of a wide array of possible architectures, including field programmable gate array (FPGA), central processing unit (CPU), application specific integrated circuits (ASIC), digital signal processor (DSP) or graphics processing unit (GPU) hardware arranged homogenously or heterogeneously. The memory 320 may be but is not limited to a random access memory (RAM), read only memory (ROM), or other electronic, optical, magnetic or any other computer readable medium.

The computing device 300 includes a communication device 340 configured to communicate with the internet 106 through one or more wireless signals. The one or more wireless signals may include Wi-Fi, Bluetooth, Zigbee, Sub-GHz RF Channel or any other wireless signal known to one of skill in the art. Alternatively, the computing device 300 may be connected to the internet 106 through a hardwired connection. The computing device 300 is configured to communicate with the environmental control device 200 through the internet 106. Communication between the computing device 300 and the environmental control device 200 may have to pass through the internet 106 and through the remote server 108. Communication may include data 120 and commands 130 of FIG. 1. The one or more environmental systems 230 are configured to receive commands 130 from the computer application 350. The one or more environmental sensors 250 are configured to transmit data 120 to the computer application 350.

The computing device 300 may include a display device 380, such as for example a computer display, an LCD display, an LED display, an OLED display, a touchscreen of a smart phone, tablet, or any other similar display device known to one of the skill in the art. A user operating the computing device 300 is able to view the computer application 350 through the display device 380.

The computing device 300 includes an input device 370 configured to receive a manual input from a user (e.g., human being) of computing device 300. The input device 370 may be a keyboard, a touch screen, a joystick, a knob, a touchpad, one or more physical buttons, a microphone configured to receive a voice command, a camera or sensor configured to receive a gesture command, an inertial measurement unit configured to detect a shake of the computing device 300, or any similar input device known to one of skill in the art. The user operating the computing device 300 is able to enter feedback into the computer application 350 through the input device 370. The input device 370 allows the user operating the computing device 300 to enter feedback into the computer application 350 via a manual input to input device 370. For example, the user may respond to a prompt on the display device 380 by entering a manual input via the input device 370. In one example, the manual input may be a touch on the touchscreen. In an embodiment, the display device 380 and the input device 370 may be combined into a single device, such as, for example, a touchscreen.

The computing device 300 device may also include a feedback device 360. The feedback device 360 may activate in response to a manual input via the input device 370. The feedback device 360 may be a haptic feedback vibration device and/or a speaker emitting a sound. The feedback device 360 device may activate to confirm that the manual input entered via the input device 370 was received via the computer application 350. For example, the feedback device 360 device may activate by emitting an audible sound or vibrate the computing device 300 to confirm that the manual input entered via the input device 370 was received via the computer application 350.

The term “about” is intended to include the degree of error associated with measurement of the particular quantity based upon the equipment available at the time of filing the application. For example, “about” can include a range of ±8% or 5%, or 2% of a given value.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

As described above, embodiments can be in the form of processor-implemented processes and devices for practicing those processes, such as processor. Embodiments can also be in the form of computer program code (e.g., computer program product) containing instructions embodied in tangible media (e.g., non-transitory computer readable medium), such as floppy diskettes, CD ROMs, hard drives, or any other non-transitory computer readable medium, wherein, when the computer program code is loaded into and executed by a computer, the computer becomes a device for practicing the embodiments. Embodiments can also be in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes a device for practicing the exemplary embodiments. When implemented on a general-purpose microprocessor, the computer program code segments configure the microprocessor to create specific logic circuits.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. An environmental control device, comprising:
a base housing;
a topper configured to be attachable and detachable from the base housing;
one or more environmental systems; and
one or more environmental sensors,
wherein the topper is shaped as a figurine.

2. The environmental control device of claim 1, wherein the base housing is translucent or transparent.

3. The environmental control device of claim 1, wherein the top is translucent or transparent.

4. The environmental control device of claim 1, wherein the one or more environmental systems comprise at a least one of:
an air filtration system;
a night light;
a humidifier;
a dehumidifier;
a speaker; or
a noise machine.

5. The environmental control device of claim 1, wherein the one or more environmental systems comprise:
an air filtration system;
a night light;
a humidifier;
a dehumidifier; and
a speaker or a noise machine.

6. The environmental control device of claim 1, wherein the one or more environmental sensors comprise at a least one of:
an air quality sensor;
a light sensor;
a humidity sensor;
a microphone;
a video camera; or
a thermometer.

7. The environmental control device of claim 1, wherein the one or more environmental sensors comprise:
an air quality sensor;
a light sensor;
a humidity sensor;
a microphone;
a video camera; and
a thermometer.

8. The environmental control device of claim 1, wherein the one or more environmental systems comprise a humidifier, wherein the topper further comprises a stationary component, and wherein the humidifier is configured to direct humidified air from the humidifier through the stationary component of the topper to make it appear that smoke is emanating from the stationary component of the topper.

9. The environmental control device of claim 1, wherein an airflow is generated by the one or more environmental system, wherein the topper further comprises a motive component, and wherein the airflow is directed to the motive component of the topper to move the motive component.

10. The environmental control device of claim 1, wherein an airflow is generated by the one or more environmental system, wherein the topper further comprises a motive component an orifice, and wherein the airflow is directed through the orifice at the motive component of the topper to move the motive component.

11. An environmental control system, comprising:
a computer application installed on a computing device or accessible via the computing device;
an environmental control device in communication with the computing device, the environmental control device comprising:
a base housing;
a topper configured to be attachable and detachable from the base housing;
one or more environmental systems, the one or more environmental systems being configured to receive commands from the computer application; and
one or more environmental sensors, the one or more environmental sensors being configured to transmit data to the computer application,
wherein the topper is shaped as a figurine.

12. The environmental control system of claim 11, wherein the base housing is translucent or transparent.

13. The environmental control system of claim 11, wherein the top is translucent or transparent.

14. The environmental control system of claim 11, wherein the one or more environmental systems comprise at a least one of:
an air filtration system;
a night light;
a humidifier;
a dehumidifier;
a speaker; or
a noise machine.

15. The environmental control system of claim 11, wherein the one or more environmental systems comprise:
an air filtration system;
a night light;
a humidifier;
a dehumidifier; and
a speaker or a noise machine.

16. The environmental control system of claim 11, wherein the one or more environmental sensors comprise at a least one of:
an air quality sensor;
a light sensor;
a humidity sensor;
a microphone;
a video camera; or
a thermometer.

17. The environmental control system of claim 11, wherein the one or more environmental sensors comprise:
an air quality sensor;
a light sensor;
a humidity sensor;
a microphone;
a video camera; and
a thermometer.

18. The environmental control system of claim 11, wherein the one or more environmental systems comprise a humidifier, wherein the topper further comprises a stationary component, and wherein the humidifier is configured to direct humidified air from the humidifier through the stationary component of the topper to make it appear that smoke is emanating from the stationary component of the topper.

19. The environmental control system of claim 11, wherein an airflow is generated by the one or more environmental system, wherein the topper further comprises a motive component, and wherein the airflow is directed to the motive component of the topper to move the motive component.

20. The environmental control system of claim 11, wherein an airflow is generated by the one or more environmental system, wherein the topper further comprises a motive component an orifice, and wherein the airflow is directed through the orifice at the motive component of the topper to move the motive component.
